(19) United States
(12) Patent Application Publication
(10) Pub. No.: US 2023/0255529 A1
(43) Pub. Date: Aug. 17, 2023

(54) Point of Care Collection and Transfer Device with Luer Lock Access Device and Syringe Compatibility

(71) Applicant: Becton, Dickinson and Company, Franklin Lakes, NJ (US)

(72) Inventors: Erica E. Neumann, Sandy, UT (US); Jonathan Karl Burkholz, Salt Lake City, UT (US)

(21) Appl. No.: 18/108,988

(22) Filed: Feb. 13, 2023

Related U.S. Application Data
(60) Provisional application No. 63/309,926, filed on Feb. 14, 2022.

(51) Int. Cl. A61B 5/15; A61M 39/10

(52) U.S. Cl. CPC A61B 5/150992; A61M 39/10

(57) ABSTRACT

An instrument advancement device includes a housing comprising a fluid path, a distal end, and a proximal end, an instrument disposed within the housing, an advancement element, where in response to movement of the advancement element with respect to the housing, the instrument is configured to advance distal to the distal end of the housing, a small sample dispense device connected to the proximal end of the housing, and a blood collection device connected to a proximal end of the small sample dispense device. The blood collection device is configured to receive an evacuated blood collection container, where the small sample dispense device is detachable from the housing and configured to dispense a blood sample.

### CROSS-REFERENCE TO RELATED APPLICATION

The present application claims priority to U.S. Provisional Application Ser. No. 63/309,926, entitled “Point of Care Collection and Transfer Device with Luer Lock Access Device and Syringe Compatibility”, filed Feb. 14, 2022, the entire disclosure of which is incorporated by reference in its entirety.

### BACKGROUND OF THE INVENTION

### Field of the Invention

The present disclosure is directed to a vascular access blood draw device and, in particular, a vascular access blood draw device with an integrated point-of-care small volume blood collection device.

### Description of Related Art

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter adapter, the catheter extending distally from the catheter adapter, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Recent developments in the peripheral intravenous catheter (PIVC) field have led to the emergence of technologies designed to facilitate blood draw and an in-dwelling PIVC. These devices have focused on the ability to reliably collect a high-quality blood sample and reduce hemolysis. The main method by which these devices work is by inserting a guide wire, probe, tube, or other instrument through the lumen of the catheter. This arrangement creates a fluid path through any thrombus or fibrin that may be occluding the tip of the catheter. A syringe or vacutainer may then be used to collect blood samples without needing to subject the patient to additional needle sticks.

Point-of-care (POC) testing includes the use of a portable testing system that can quickly deliver results using small volume blood samples, such as for glucose testing. Blood samples are typically collected via finger pricks or through existing vascular access. However, the majority of vascular access devices are not optimized for collection of small blood samples. The current field is moving toward miniaturized and personalized healthcare with the goal of POC testing that can provide rapid diagnostic results with a single drop of blood. There is a current need to provide POC blood sample collection options from a PIVC in anticipation for increased POC diagnostic testing.

Infusion and blood withdrawal using the catheter may be difficult for several reasons, particularly when an indwelling time of the catheter increases. A fibrin sheath or thrombus may form on an internal surface of the catheter assembly, an external surface of the catheter assembly, or within the vasculature near the distal tip of the catheter. The fibrin sheath or thrombus may block or narrow a fluid pathway through the catheter, which may impair infusion and/or collection of a high-quality blood sample.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

In one aspect or embodiment, an instrument advancement device includes a housing comprising a fluid path, a distal end, and a proximal end, an instrument disposed within the housing, an advancement element, wherein in response to movement of the advancement element with respect to the housing, with the instrument configured to advance distal to the distal end of the housing, a small sample dispense device connected to the proximal end of the housing, and a blood collection device connected to a proximal end of the small sample dispense device. The blood collection device is configured to receive an evacuated blood collection container, where the small sample dispense device is detachable from the housing and configured to dispense a blood sample.

The small sample dispense device may be configured to receive a volume of 0.3 to 500 microliters. The small sample dispense device may be configured to receive a volume of 0.3 to 100 microliters.

The small sample dispense device may be formed integrally with the blood collection device. The small sample dispense device may be removably attached to the blood collection device. The small sample dispense device may include a needle-free connector configured to be connected to the blood sample collection device.

The small sample dispense device may be configured to be squeezed to dispense fluid from the small sample dispense device. The small sample dispense device may be configured to be attached to a syringe barrel to dispense fluid from the small sample dispense device.

In a further aspect or embodiment, a blood sample collection system includes a vascular access device, a small sample dispense device connected to the vascular access device, and a blood collection device connected to a proximal end of the small sample dispense device. The blood collection device is configured to receive an evacuated blood collection container, where the small sample dispense device is detachable from the vascular access device and configured to dispense a blood sample.

The vascular access device may be a catheter adapter including a catheter and a patient connector, with the small sample dispense device connected to the patient connector. The patient connector may be connected to the catheter adapter via tubing. The vascular access device may include extension tubing.

The small sample dispense device may be vented to provide a blood flashback during use of the vascular access device.

### BRIEF DESCRIPTION OF THE DRAWINGS

Example embodiments will be described and explained with additional specificity and detail through the use of the accompanying drawings in which:

FIG. 1A is an upper perspective view of an example instrument advancement device, illustrating an example advancement element in an example initial or retracted position, according to some embodiments;

FIG. 1B is a longitudinal cross-sectional view of the instrument advancement device of FIG. 1A;

FIG. 1C is a longitudinal cross-sectional view of the instrument advancement device of FIG. 1A, illustrating the advancement element in the initial or retracted position according to some embodiments;

FIG. 1D is a cross-sectional view of the instrument advancement device of FIG. 1A along the line 1D-1D of FIG. 1A according to some embodiments;

FIG. 1E is an enlarged view of a portion of FIG. 1D according to some embodiments;

FIG. 1F is a cross-sectional view of the instrument advancement device of FIG. 1A along the line 1F-1F of FIG. 1A according to some embodiments;

FIG. 2 is a perspective view of an instrument advancement device and blood sample collection device according to one non-limiting example or embodiment of the present disclosure;

FIG. 3 is side view of an instrument advancement device and blood sample collection device according to one non-limiting example or embodiment of the present disclosure;

FIG. 4 is a perspective view of a vascular access device and a blood sample collection device according to one non-limiting example or embodiment of the present disclosure; and

FIG. 5 is a perspective view of the blood sample collection device of FIG. 4 dispensing a small volume blood sample to a blood test cartridge.

### DESCRIPTION OF THE DISCLOSURE

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates, and grammatical variants of those words or phrases.

The figures accompanying this application are representative in nature, and should not be construed as implying any particular scale or directionality, unless otherwise indicated. For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations and step sequences, except where expressly specified to the contrary. Hence, specific dimensions and other physical characteristics related to the embodiments disclosed herein are not to be considered as limiting.

The present disclosure is directed to a vascular access instrument delivery device that provides needle-free delivery of a probe or tube to a patient's vascular system for blood collection with integrated small volume blood sample collection devices for POC testing. These blood sample collection containers may be optimized to dispense blood to various POC systems and instruments, including cartridges for POC systems or test strips such as for glucose monitors.

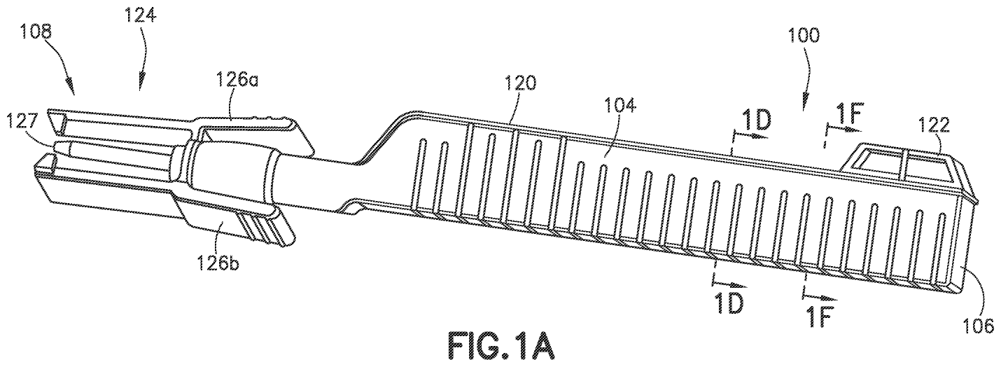

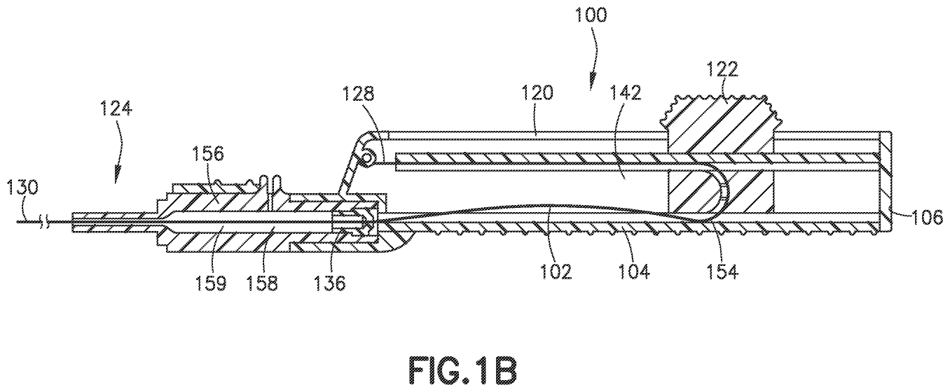

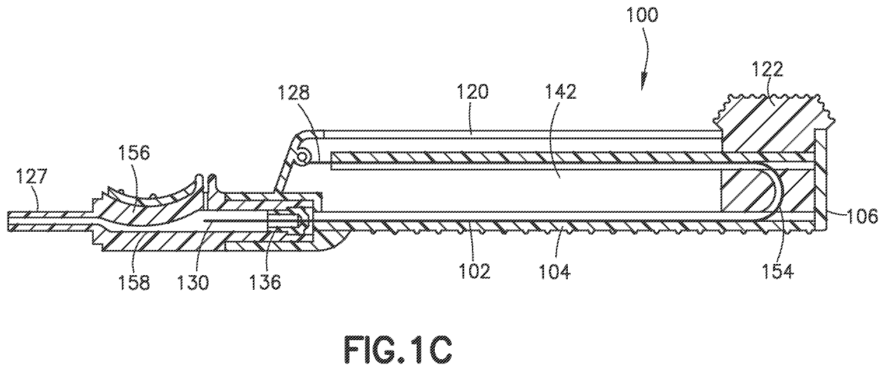

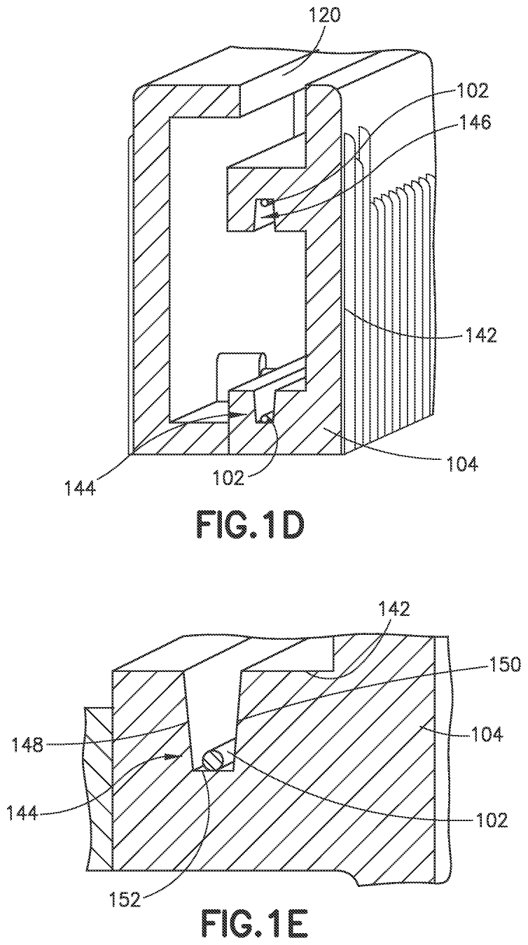

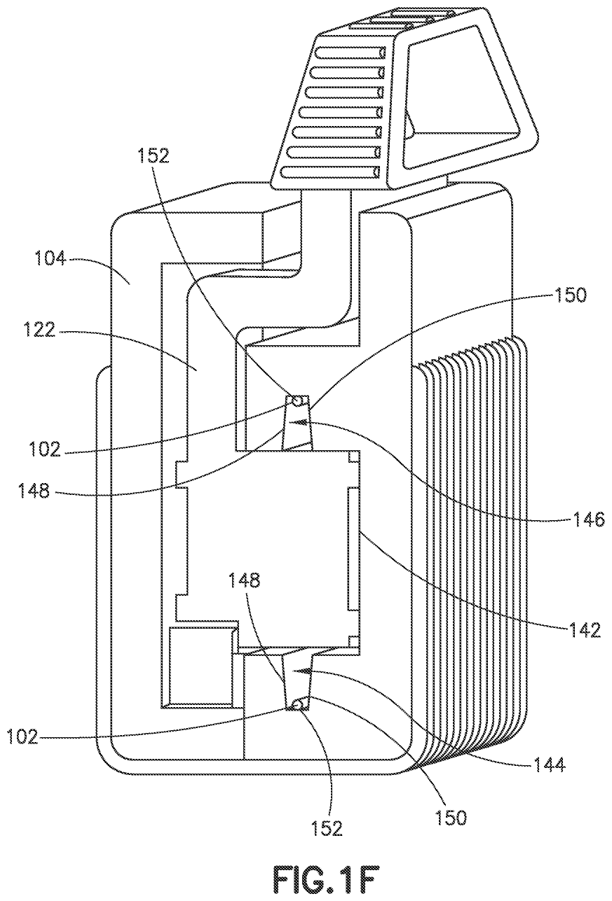

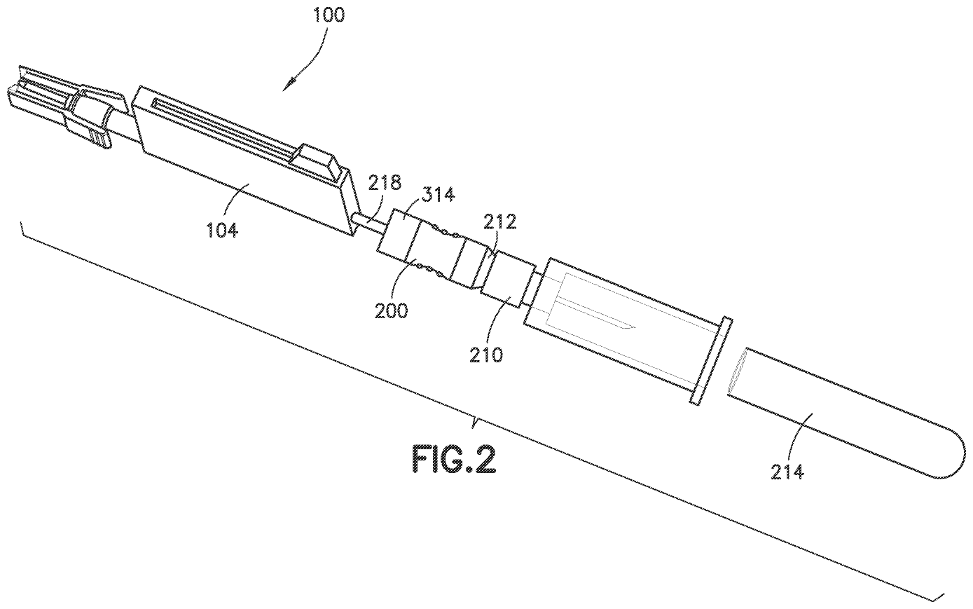

Referring now to FIGS. 1A-1F, in some embodiments, an instrument advancement device 100 may be configured to deliver an instrument 102 through a catheter of a catheter assembly. In some embodiments, the instrument 102 may be advanced through the catheter to push past any occlusions in the catheter or vasculature (e.g., thrombus or fibrin sheath at a tip of the catheter, vein collapse, valves, etc.) to create a clear pathway for fluid flow. In some embodiments, the instrument 102 may reduce or remove occlusions, improving patency of the catheter for medication and fluid delivery, as well as blood acquisition, during a dwell time of the catheter.

In some embodiments, the instrument 102 may include a guidewire, a probe, a guidewire or a probe with one or more sensors, or another suitable instrument. In some embodiments, the sensors may be used for patient or device monitoring and may include sensors measuring pressure, temperature, pH, blood chemistry, oxygen saturation, flow rate, or another physiological property. The instrument 102 may be a flow tube or other suitable blood draw instrument. In some embodiment, the instrument advancement 100 device may be the PIVO™ blood draw device commercially available from Velano Vascular. In one embodiment, the instrument advancement device is the same or similar to the blood draw device shown in U.S. Pat. No. 11,090,461, which is hereby incorporated by reference in its entirety.

In some embodiments, the catheter may include a peripheral IV catheter, a peripherally-inserted central catheter, or a midline catheter. In some embodiments, the catheter through which the instrument 102 may be delivered, may have been previously inserted into vasculature of a patient and may be dwelling within the vasculature when the instrument 102 is advanced through the catheter.

In some embodiments, the instrument 102 may be disposed within a housing 104, which may be configured to protect the instrument 102 from damage and/or contamination from a surrounding external environment. In some embodiments, the housing 104 may be rigid or semi-rigid. In some embodiments, the housing 104 may be made of one or more of stainless steel, aluminum, polycarbonate, metal, ceramic, plastic, and another suitable material. In some embodiments, the housing 104 may include a proximal end 106, a distal end 108, and a slot 120. In some embodiments, the slot 120 may extend parallel to a longitudinal axis of the housing 104.

In some embodiments, the instrument advancement device 100 may include an advancement element 122, which may extend through the slot 120 and may be configured to move linearly along the slot 120 between a retracted position illustrated, for example, in FIG. 1A, and an advanced position. In some embodiments, the user may pinch or grasp the advancement element 122 to move the advancement element 122 between the retracted position and the advanced position.

In some embodiments, the distal end 108 of the housing 104 may include a distal connector 124. In some embodiments, the distal connector 124 may include opposing lever arms 126a, 126b. In some embodiments, distal ends of the opposing lever arms 126a, 126b may be configured to move apart from each other in response to pressure applied to proximal ends of the opposing lever arms 126a, 126b. In some embodiments, in response to removal of the pressure applied to the proximal ends of the opposing lever arms 126a, 126b, the distal ends may move closer to each other and clasp a portion of the catheter assembly, such as a needleless connector, another connector, or a proximal end of a catheter adapter, for example. In some embodiments, the distal connector 124 may include a blunt cannula 127 or a male luer configured to insert into the portion of the catheter assembly.

In some embodiments, the distal connector 124 may include any suitable connector. For example, the distal connector 124 may include a threaded male luer, a slip male luer, a threaded male luer with a spin lock, a threaded male luer with a removable blunt cannula snap connection, a slip male luer with a removable blunt cannula snap connection, or another suitable connector. In some embodiments, the distal connector 124 may include one or more bond pockets, which may each be configured to receive an extension tube. In some embodiments, the distal connector 124 may be monolithically formed as a single unit with a body of the housing 104 that includes the slot 120.

In some embodiments, the instrument 102 may include a first end 128 and a second end 130. In some embodiments, movement of the advancement element 122 from the retracted position to the advanced position may cause the second end 130 of the instrument 102 to be advanced beyond the distal end 108 of the housing 104. In some embodiments, moving the advancement element 122 to the advanced position may introduce the instrument 102 into the catheter assembly and/or through the catheter. In some embodiments, in response to the instrument 102 being introduced into the catheter assembly and/or through the catheter, the instrument 102 may access a fluid pathway of the catheter assembly and/or the vasculature of a patient.

In some embodiments, the catheter of the catheter assembly with significant dwelling time within the vasculature may be susceptible to narrowing, collapse, kinking, blockage by debris (e.g., fibrin or platelet clots), and adhering of a tip of the catheter to the vasculature. Thus, blood withdrawal using the catheter may be difficult. In some embodiments, the instrument 102 may have a diameter less than a diameter of the catheter of the catheter assembly to provide access to the vasculature of the patient without any additional needle sticks. In some embodiments, the instrument 102 may clear the pathway for collecting a blood sample. Thus, in some embodiments, the instrument advancement device 100 may be used for needle-free blood collection and/or fluid infusion.

In some embodiments, an extension tube may be coupled to the instrument advancement device 100, and the extension tube may be used for blood collection and/or fluid infusion. In some embodiments, the extension tube may extend from a port of the housing 104. In some embodiments, a septum 136 may be within the housing 104 to enable the instrument 102 to advance and/or retract while maintaining a closed fluid path. In some embodiments, the instrument 102 may be configured to extend through the septum 136. In some embodiments, the septum 136 may be disposed proximal to a port and distal to the advancement element 122 in the advanced position. In some embodiments, the septum 136 may include silicone, rubber, an elastomer, or another suitable material. In some embodiments, the septum 136 may include an aperture, slit, or the like to accommodate the instrument 102 therethrough.

In some embodiments, an inner surface 142 of the housing 104 may include one or more grooves. For example, the inner surface 142 may include a first groove 144 and/or a second groove 146. In some embodiments, the first groove 144 and/or the second groove 146 may be disposed within the housing 104 between the proximal end 106 and the distal end 108. In some embodiments, the instrument 102 may be disposed within the first groove 144 and/or the second groove 146. In some embodiments, the first groove 144 and/or the second groove 146 may include a support wall 148, another support wall 150 opposite the support wall 148, and a bottom 152 extending between the support wall 148 and the other support wall 150. In some embodiments, the first groove 144 and/or the second groove 146 may be open opposite the bottom 152. In some embodiments, the first groove 144 and/or the second groove 146 may be linear and/or configured to guide the instrument 102 as the instrument 102 is advanced distally and/or retracted proximally.

In some embodiments, the advancement element 122 may include an arc-shaped channel 154, which may be U-shaped. In some embodiments, the instrument 102 may extend and move through the arc-shaped channel 154. In some embodiments, the first end 128 of the instrument 102 may be fixed. In some embodiments, the first end 128 of the instrument may be fixed within the housing 104. In some embodiments, in response to movement of the advancement element 122 a first distance, the second end 130 of the instrument 102 may be configured to advance distally a second distance. In some embodiments, the second distance may be twice the first distance. In some embodiments, the second distance may be more than twice the first distance. In these and other embodiments, the instrument 102 may extend through multiple U-shapes or other arc-shapes. In some embodiments, the distal end 108 of the housing 104 may include a compressible portion 156 proximate a chamber 158 configured to hold blood. In some embodiments, the compressible portion 156 may be constructed of a flexible material, which may have a lower durometer than portions of the housing 104 surrounding the compressible portion 156. In some embodiments, a blood collection pathway 159 may extend within the chamber 158 and/or through the extension tube 132.

In some embodiments, because the first groove 144 and/or the second groove 146 are open opposite the bottom 152, the instrument 102 may tend to buckle in response to the advancement element 122 being advanced distally, as illustrated, for example, in FIG. 1B. Thus, in some embodiments, one or more support features may be used. The support features may be described further in U.S. patent application No. 63/164,976, filed Mar. 23, 2021, entitled VASCULAR ACCESS DEVICE TO REDUCE BUCKLING OF AN INSTRUMENT, which is herein incorporated by reference in its entirety.

With reference to FIG. 2, according to one non-limiting embodiment or aspect of the present disclosure, the instrument advancement device 100 may include additional features for drawing blood from the blood path of the patient. In this embodiment, a small sample dispense device 200 is connected to a fluid path 218 of the instrument advancement device 100, and a blood collection device 210 is connected to a proximal end 212 of the small sample dispense device 200. The blood collection device 210 is configured to receive an evacuated blood collection container 214, such as a BD Vacutainer® blood collection tube. The small sample dispense device 200 is detachable from the housing 104 and configured to dispense a blood sample. In one aspect or embodiment, the blood collection device 210 is a BD Vacutainer® Luer-Lok™ access device. The instrument advancement device 100 and the small sample dispense device 200 may be configured to dispense small volume blood samples to POC systems, such as blood cartridges or test strips, which may be used in glucose monitors. Although the small sample dispense device 200 is shown in connection with the instrument advancement device 100, the small sample dispense device 200 may be used directly with a catheter or catheter adapter.

In one aspect or embodiment, the small sample dispense device 200 is configured to receive a volume of 0.3 to 500 microliters. In one aspect or embodiment, the small sample dispense device 200 is configured to receive a volume of 0.3 to 100 microliters.

As shown in FIG. 2, in one aspect or embodiment, the small sample dispense device 200 is formed integrally with the blood collection device 210.

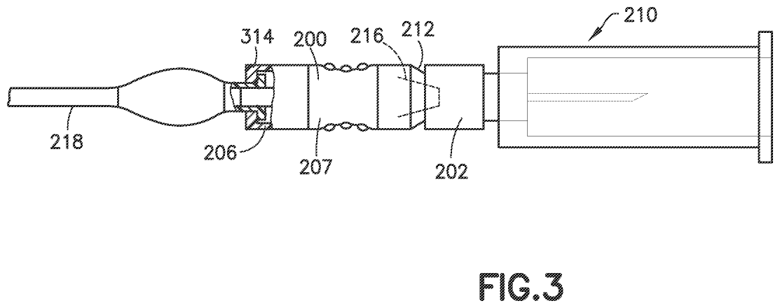

As shown in FIG. 3, in a further aspect or embodiment, the small sample dispense device 200 is removably attached to the blood collection device 210. When removable from the blood collection device 210, the small sample dispense device 200 may include a needle-free connector 216, as shown in FIG. 3, that is configured to be connected to the blood sample collection device 210. The needle-free connector 216 is configured to prevent fluid from leaving the small sample dispense device 200 when detached from the blood sample collection device 210.

The small sample dispense device 200 may be fluidly connected to the fluid path 218 of the instrument 102 of the instrument advancement device 100, which may be an extension set having a connector or a flow tube. The instrument advancement device 100 may be used to draw a small volume blood sample from the patient into the blood sample collection device 210. The small volume blood sample may then be dispensed onto a POC cartridge after all evacuated collection container draws are complete to ensure a non-contaminated blood sample. The blood droplets of the small volume blood sample may be deposited on a blood cartridge for analysis and testing by a POC testing system.

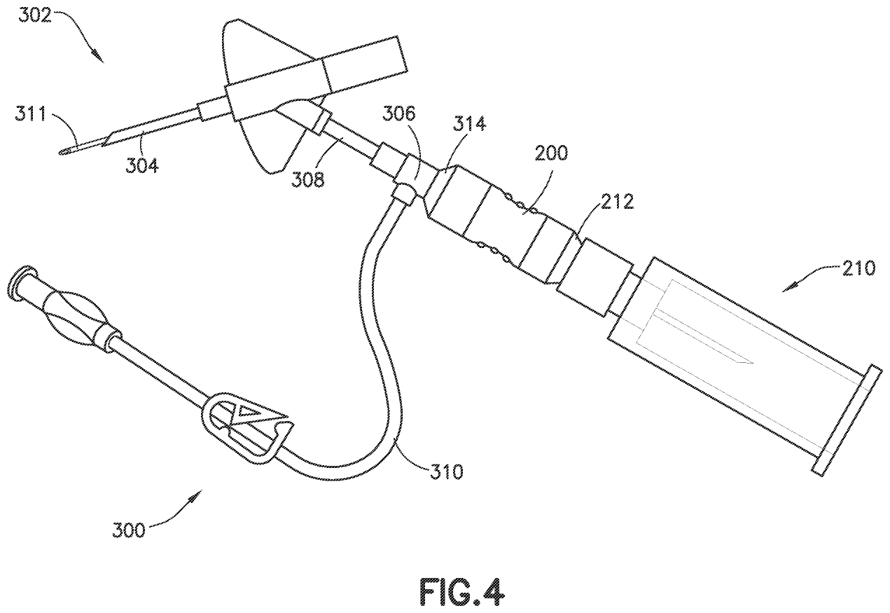

As shown in FIG. 4, a blood sample collection system 300 according to one aspect or embodiment of the present application is shown. The blood sample collection system 300 includes a vascular access device 302, the small sample dispense device 200 connected to the vascular access device 302, and the blood collection device 210 connected to the proximal end 212 of the small sample dispense device 200. The small sample dispense device 200 is detachable from the vascular access device 302 and configured to dispense a blood sample. In one aspect or embodiment, the vascular access device 302 is a catheter adapter including a catheter 304 and a patient connector 306, with the small sample dispense device 200 connected to the patient connector 306. The patient connector 306 may be connected to the catheter adapter via tubing 308. In one aspect or embodiment, the vascular access device 302 includes an extension tubing set 310. The vascular access device 302 may further include a needle 311 or needle insertion assembly (not shown) configured to place the catheter 304 within a patient's vasculature.

As discussed above, the small sample dispense device 200 may be formed integrally with the blood collection device 210 or may be detachable from the blood collection device 210. The small sample dispense device 200 is configured to be in line with the fluid path of the vascular access device 302 and configured to be removed after an evacuated blood collection container or syringe blood draw are complete thereby ensuring any waste volume has been removed from the system 300.

In one aspect or embodiment, the small sample dispense device 200 is vented to provide a blood flashback during use of the vascular access device 302.

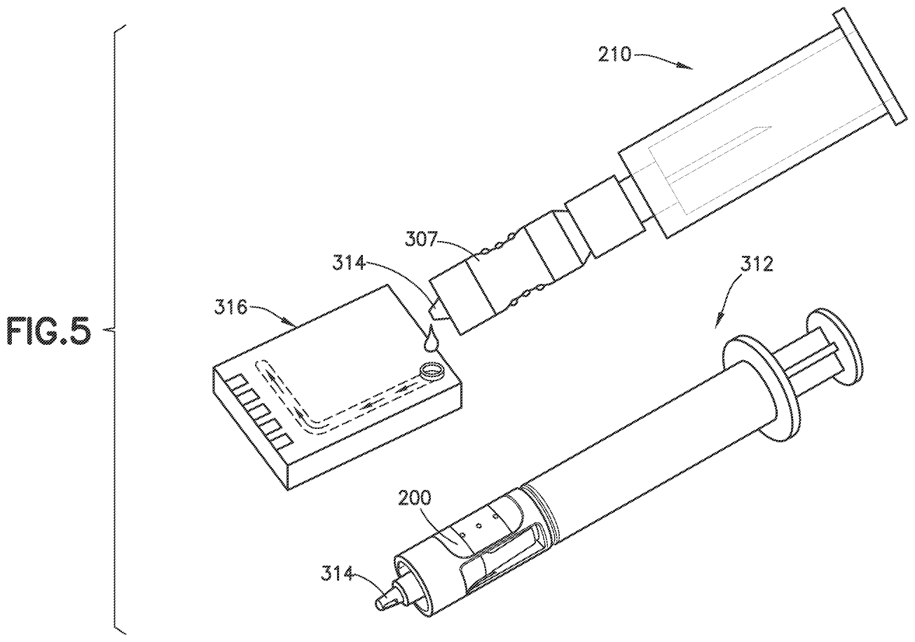

As shown in FIG. 5, the small sample dispense device 200 is configured to be squeezed to dispense fluid from the small sample dispense device 200 or, alternatively, the small sample dispense device 200 is configured to be attached to a syringe barrel 312 to dispense fluid from the small sample dispense device 200. The small sample dispense device 200 may be used to dispense a blood sample to a POC system 316, including cartridges, test strips, or any other suitable arrangement. In one aspect or embodiment, a sample is dispensed from a distal end 314 of the small sample dispense device 200.

In one aspect or embodiment, the small sample dispense device 200 includes fluid path geometry configured to prevent hemolysis of the blood sample. In one aspect or embodiment, the fluid path geometry lowers a flow rate of blood within the fluid pathway, which may, in turn, lower a shear rate for hemolysis management. The fluid path of the small sample dispense device 200 may have an inner diameter, length, and/or undulations configured to provide a predetermined flow rate configured to minimize or prevent hemolysis of the blood sample. In some aspects or embodiments, the small sample dispense device 200 includes one of the flow path geometries shown and described in U.S. Patent Application Publication No. 2021/10186394, which is hereby incorporated by reference in its entirety.

While the present invention has been described in terms of the above detailed description, those of ordinary skill in the art will understand that alterations may be made within the spirit of the invention.

The invention claimed is:

1. An instrument advancement device comprising:
a housing comprising a fluid path, a distal end, and a proximal end;
an instrument disposed within the housing;
an advancement element, wherein in response to movement of the advancement element with respect to the housing, the instrument is configured to advance distal to the distal end of the housing;
a small sample dispense device connected to the proximal end of the housing; and
a blood collection device connected to a proximal end of the small sample dispense device, the blood collection device is configured to receive an evacuated blood collection container, wherein the small sample dispense device is detachable from the housing and configured to dispense a blood sample.

2. The instrument advancement device of claim 1, wherein the small sample dispense device is configured to receive a volume of 0.3 to 500 microliters.

3. The instrument advancement device of claim 2, wherein the small sample dispense device is configured to receive a volume of 0.3 to 100 microliters.

4. The instrument advancement device of claim 1, wherein the small sample dispense device is formed integrally with the blood collection device.

5. The instrument advancement device of claim 1, wherein the small sample dispense device is removably attached to the blood collection device.

6. The instrument advancement device of claim 5, wherein the small sample dispense device comprises a needle-free connector configured to be connected to the blood sample collection device.

7. The instrument advancement device of claim 1, wherein the small sample dispense device is configured to be squeezed to dispense fluid from the small sample dispense device.

8. The instrument advancement device of claim 1, wherein the small sample dispense device is configured to be attached to a syringe barrel to dispense fluid from the small sample dispense device.

9. A blood sample collection system comprising:
a vascular access device;
a small sample dispense device connected to the vascular access device; and
a blood collection device connected to a proximal end of the small sample dispense device, the blood collection device is configured to receive an evacuated blood collection container, wherein the small sample dispense device is detachable from the vascular access device and configured to dispense a blood sample.

10. The blood sample collection system of claim 9, wherein the vascular access device comprises a catheter adapter including a catheter and a patient connector, the small sample dispense device connected to the patient connector.

11. The blood sample collection system of claim 10, wherein the patient connector is connected to the catheter adapter via tubing.

12. The blood sample collection system of claim 10, wherein the vascular access device comprises extension tubing.

13. The blood sample collection system of claim 9, wherein the small sample dispense device is configured to receive a volume of 0.3 to 500 microliters.

14. The blood sample collection system of claim 9, wherein the small sample dispense device is configured to receive a volume of 0.3 to 100 microliters.

15. The blood sample collection system of claim 9, wherein the small sample dispense device is formed integrally with the blood collection device.

16. The blood sample collection system of claim 9, wherein the small sample dispense device is removably attached to the blood collection device.

17. The blood sample collection system of claim 16, wherein the small sample dispense device comprises a needle-free connector configured to be connected to the blood sample collection device.

18. The blood sample collection system of claim 9, wherein the small sample dispense device is configured to be squeezed to dispense fluid from the small sample dispense device.

19. The blood sample collection system of claim 9, wherein the small sample dispense device is configured to be attached to a syringe barrel to dispense fluid from the small sample dispense device.

20. The blood sample collection system of claim 9, wherein the small sample dispense device is vented to provide a blood flashback during use of the vascular access device.
